SEQUENCE LISTING (116 sequences)

SEQ ID NO: 1 - protein, 9 aa, Artificial Sequence
  Ala substituted varient of a human sequence
AAAPKIFYA

SEQ ID NO: 2 - protein, 9 aa, Artificial sequence
  Ala substituted varient of a human sequence
AAGPKIFYA

SEQ ID NO: 3 - protein, 9 aa, Artificial sequence
  Ala substituted varient of a human sequence
AAAAKIFYA

SEQ ID NO: 4 - protein, 9 aa, Artificial sequence
  Ala substituted varient of a human sequence
AAAPAIFYA

SEQ ID NO: 5 - protein, 9 aa, Artificial sequence
  Ala substituted varient of a human sequence
AAAPKAFYA

SEQ ID NO: 6 - protein, 9 aa, Artificial Sequence
  Ala substituted varient of a human sequence
AAAPKIFAA

SEQ ID NO: 7 - protein, 9 aa, Artificial sequence
  Ala substituted varient of a human sequence
AADPKIFYA

SEQ ID NO: 8 - protein, 9 aa, Artificial sequence
  Ala substituted varient of a human sequence
AADGKIFYA

SEQ ID NO: 9 - protein, 9 aa, Artificial sequence
  Ala subsstituted varient of a human sequence
AADEKIFYA

SEQ ID NO: 10 - protein, 9 aa, Artificial sequence
  Ala substituted varient of a human sequence
AAGGKIFYA

SEQ ID NO: 11 - protein, 9 aa, Artificial sequence
  Ala substituted varient of a human sequence
AAGEKIFYA

SEQ ID NO: 12 - protein, 9 aa, Artificial sequence
  Ala substituted varient of a human sequence
ALAPKIFYA

SEQ ID NO: 13 - protein, 9 aa, Artificial sequence
  Ala substituted varient of a human sequence
AMAPKIFYA

SEQ ID NO: 14 - protein, 9 aa, Artificial sequence
  Ala substituted varient of a human sequence
AAAPKIFYL

SEQ ID NO: 15 - protein, 9 aa, Artificial sequence
  Ala substituted varient of a human sequence
AAAPKIAYA

SEQ ID NO: 16 - protein, 9 aa, Homo sapiens
KASEKIFYV

SEQ ID NO: 17 - protein, 9 aa, Artificial sequence
  Ala substituted varient of a human sequence
AASEKIFYV

SEQ ID NO: 18 - protein, 9 aa, artificial sequence
  Ala substituted varient of a human sequence
KAAEKIFYV

SEQ ID NO: 19 - protein, 9 aa, Artificial sequence
  Ala substituted varient of a human sequence
KASAKIFYV

SEQ ID NO: 20 - protein, 9 aa, Artificial sequence
  Ala substituted varient of a human sequence
KASEAIFYV

SEQ ID NO: 21 - protein, 9 aa, Artificial sequence
  Ala substituted varient of a human sequence
KASEKAFYV

SEQ ID NO: 22 - protein, 9 aa, Artificial sequence
  Ala substituted varient of a human sequence
KASEKIAYV

SEQ ID NO: 23 - protein, 9 aa, Artificial sequence
  Ala substituted varient of a human sequence
KASEKIFAV

SEQ ID NO: 24 - protein, 9 aa, Artificial sequence
  Ala substituted varient of a human sequence
KASEKIFYA

SEQ ID NO: 25 - protein, 9 aa, Homo sapiens
YMDGTMSQV

SEQ ID NO: 26 - protein, 9 aa, Homo sapiens
MLLAVLYCL

SEQ ID NO: 27 - protein, 10 aa, Homo sapiens
ELAGIGILTV

SEQ ID NO: 28 - protein, 9 aa, Homo sapiens
IMPKAGLLI

SEQ ID NO: 29 - protein, 9 aa, Homo sapiens
FLWGPRALV

SEQ ID NO: 30 - protein, 9 aa, Homo sapiens
VRIGHLYIL

SEQ ID NO: 31 - protein, 10 aa, Homo sapiens
YLQLVFGIEV

SEQ ID NO: 32 - protein, 9 aa, Homo sapiens
FLWGPRALV

SEQ ID NO: 33 - protein, 10 aa, Homo sapiens
  Xaa 10: Xaa may be Val or may be absent
VLPDVFIRCX

SEQ ID NO: 34 - protein, 9 aa, Homo sapiens
GLYDGMEHL

SEQ ID NO: 35 - protein, 10 aa, Homo sapiens
MEVDPIGHLY

SEQ ID NO: 36 - protein, 9 aa, Homo sapiens
EVDPIGHLY

SEQ ID NO: 37 - protein, 9 aa, Homo sapiens
SLLMWITQC

SEQ ID NO: 38 - protein, 9 aa, Homo sapiens
IMPKAGLLI

SEQ ID NO: 39 - protein, 9 aa, Homo sapiens
EVDPIGHLY

SEQ ID NO: 40 - protein, 10 aa, Homo sapiens
GVYDGREHTV

SEQ ID NO: 41 - protein, 9 aa, Homo sapiens
EADPTGHSY

SEQ ID NO: 42 - protein, 9 aa, Homo sapiens
SEIWRDIDF

SEQ ID NO: 43 - protein, 9 aa, Homo sapiens
LPSSADVEF

SEQ ID NO: 44 - protein, 9 aa, Homo sapiens
SAYGEPRKL

SEQ ID NO: 45 - protein, 8 aa, Homo sapiens
YRPRPRRY

SEQ ID NO: 46 - protein, 9 aa, Homo sapiens
LAMPFATPM

SEQ ID NO: 47 - protein, 9 aa, Homo sapiens
ARGPESRLL

SEQ ID NO: 48 - protein, 9 aa, Homo sapiens
YYWPRPRRY

SEQ ID NO: 49 - protein, 9 aa, Homo sapiens
AARAVFLAL

SEQ ID NO: 50 - protein, 12 aa, Homo sapiens
TQHFVQENYLEY

SEQ ID NO: 51 - protein, 11 aa, Homo sapiens
SLLMWITQCFL

SEQ ID NO: 52 - protein, 14 aa, Homo sapiens
AELVHFLLLKYRAR

SEQ ID NO: 53 - protein, 14 aa, Homo sapiens
LLKYRAREPVTKAE

SEQ ID NO: 54 - protein, 14 aa, Homo sapiens
AELVHFLLLKYRAR

SEQ ID NO: 55 - protein, 12 aa, Homo sapiens
EYVIKVSARVRF

SEQ ID NO: 56 - protein, 14 aa, Homo sapiens
LLKYRAREPVTKAE

SEQ ID NO: 57 - protein, 20 aa, Homo sapiens
PGVLLKEFTVSGNILTIRLT

SEQ ID NO: 58 - protein, 18 aa, Homo sapiens
AADHRQLQLSISSCLQQL

SEQ ID NO: 59 - protein, 9 aa, Artificial
  Peptides with nine amino acids wherein two of the following criteria in is satisfied Lys at position 5; Phe at position 7; or Tyr at position 8
AAAPKQFLA

SEQ ID NO: 60 - protein, 9 aa, Artificial
  Peptides with nine amino acids wherein two of the following criteria in is satisfied Lys at position 5; Phe at position 7; or Tyr at position 8
AASPKSFTL

SEQ ID NO: 61 - protein, 9 aa, Artificial
  Peptides with nine amino acids wherein two of the following criteria in is satisfied Lys at position 5; Phe at position 7; or Tyr at position 8
VSAPKVFQA

SEQ ID NO: 62 - protein, 9 aa, Artificial
  Peptides with nine amino acids wherein two of the following criteria in is satisfied Lys at position 5; Phe at position 7; or Tyr at position 8
IASPKATYV

SEQ ID NO: 63 - protein, 9 aa, Artificial
  Peptides with nine amino acids wherein two of the following criteria in is satisfied Lys at position 5; Phe at position 7; or Tyr at position 8
VSAPKIFQA

SEQ ID NO: 64 - protein, 9 aa, Artificial
  Peptides with nine amino acids wherein two of the following criteria in is satisfied Lys at position 5; Phe at position 7; or Tyr at position 8
NSLPKVAYA

SEQ ID NO: 65 - protein, 9 aa, Artificial
  Peptides with nine amino acids wherein two of the following criteria in is satisfied Lys at position 5; Phe at position 7; or Tyr at position 8
ASLPKVSYV

SEQ ID NO: 66 - protein, 9 aa, Artificial
  Peptides with nine amino acids wherein two of the following criteria in is satisfied Lys at position 5; Phe at position 7; or Tyr at position 8
KAEPKAPYA

SEQ ID NO: 67 - protein, 9 aa, Artificial
  Peptides with nine amino acids wherein two of the following criteria in is satisfied Lys at position 5; Phe at position 7; or Tyr at position 8
HSLPKVSYA

SEQ ID NO: 68 - protein, 9 aa, Artificial
  Peptides with nine amino acids wherein two of the following criteria in is satisfied Lys at position 5; Phe at position 7; or Tyr at position 8
TASPKEFYA

SEQ ID NO: 69 - protein, 9 aa, Artificial
  Peptides with nine amino acids wherein two of the following criteria in is satisfied Lys at position 5; Phe at position 7; or Tyr at position 8
ASVPKELYL

SEQ ID NO: 70 - protein, 9 aa, Artificial
  Peptides with nine amino acids wherein two of the following criteria in is satisfied Lys at position 5; Phe at position 7; or Tyr at position 8
SPDPKICYV

SEQ ID NO: 71 - protein, 9 aa, Artificial
  Peptides with nine amino acids wherein two of the following criteria in is satisfied Lys at position 5; Phe at position 7; or Tyr at position 8
VAEPKESYV

SEQ ID NO: 72 - protein, 9 aa, Artificial
  Peptides with nine amino acids wherein two of the following criteria in is satisfied Lys at position 5; Phe at position 7; or Tyr at position 8
ISAPKIFRV

SEQ ID NO: 73 - protein, 9 aa, Artificial
  Peptides with nine amino acids wherein two of the following criteria in is satisfied Lys at position 5; Phe at position 7; or Tyr at position 8
AVYPKIFYV

SEQ ID NO: 74 - protein, 9 aa, Artificial
  Peptides with nine amino acids wherein two of the following criteria in is satisfied Lys at position 5; Phe at position 7; or Tyr at position 8
KASEKIIYV

SEQ ID NO: 75 - protein, 9 aa, Artificial
  Peptides with nine amino acids wherein two of the following criteria in is satisfied Lys at position 5; Phe at position 7; or Tyr at position 8
VQDPKVTYL

SEQ ID NO: 76 - protein, 9 aa, Artificial
  Peptides with nine amino acids wherein two of the following criteria in is satisfied Lys at position 5; Phe at position 7; or Tyr at position 8
CPVPKIFYV

SEQ ID NO: 77 - protein, 9 aa, Artificial
  Peptides with nine amino acids wherein two of the following criteria in is satisfied Lys at position 5; Phe at position 7; or Tyr at position 8
FAKPKITYV

SEQ ID NO: 78 - protein, 9 aa, Artificial
  Peptides with nine amino acids wherein two of the following criteria in is satisfied Lys at position 5; Phe at position 7; or Tyr at position 8
QTPPKIDYL

SEQ ID NO: 79 - protein, 9 aa, Artificial
  Peptides with nine amino acids wherein two of the following criteria in is satisfied Lys at position 5; Phe at position 7; or Tyr at position 8
LRSPKLFYA

SEQ ID NO: 80 - protein, 9 aa, Artificial
  Peptides with nine amino acids wherein two of the following criteria in is satisfied Lys at position 5; Phe at position 7; or Tyr at position 8
KYSEKISYV

SEQ ID NO: 81 - protein, 9 aa, Artificial
  Peptides with nine amino acids wherein two of the following criteria in is satisfied Lys at position 5; Phe at position 7; or Tyr at position 8
EQKPKDFYA

SEQ ID NO: 82 - protein, 9 aa, Artificial
  Peptides with nine amino acids wherein two of the following criteria in is satisfied Lys at position 5; Phe at position 7; or Tyr at position 8
RTTPKDFYV

SEQ ID NO: 83 - protein, 9 aa, Artificial
  Peptides with nine amino acids wherein two of the following criteria in is satisfied Lys at position 5; Phe at position 7; or Tyr at position 8
RADPKHKYL

SEQ ID NO: 84 - protein, 9 aa, Artificial
  Peptides with nine amino acids wherein two of the following criteria in is satisfied Lys at position 5; Phe at position 7; or Tyr at position 8
KRGWKTFYA

SEQ ID NO: 85 - protein, 9 aa, Artificial
  Peptides with nine amino acids wherein two of the following criteria in is satisfied Lys at position 5; Phe at position 7; or Tyr at position 8
TSSWKKFYL

SEQ ID NO: 86 - protein, 9 aa, Artificial
  Peptides with nine amino acids wherein two of the following criteria in is satisfied Lys at position 5; Phe at position 7; or Tyr at position 8
LSRPKRFYL

SEQ ID NO: 87 - protein, 9 aa, Artificial
  Peptides with nine amino acids wherein two of the following criteria in is satisfied Lys at position 5; Phe at position 7; or Tyr at position 8
NTYPKGFYC

SEQ ID NO: 88 - protein, 9 aa, Artificial
  Peptides with nine amino acids wherein two of the following criteria in is satisfied Lys at position 5; Phe at position 7; or Tyr at position 8
SKLPKDFYD

SEQ ID NO: 89 - protein, 9 aa, Artificial
  Peptides with nine amino acids wherein two of the following criteria in is satisfied Lys at position 5; Phe at position 7; or Tyr at position 8
ALSPKEFYE

SEQ ID NO: 90 - protein, 9 aa, Artificial
  Peptides with nine amino acids wherein two of the following criteria in is satisfied Lys at position 5; Phe at position 7; or Tyr at position 8
KVDPKPFYE

SEQ ID NO: 91 - protein, 9 aa, Artificial
  Peptides with nine amino acids wherein two of the following criteria in is satisfied Lys at position 5; Phe at position 7; or Tyr at position 8
VGSEKLFYE

SEQ ID NO: 92 - protein, 9 aa, Artificial
  Peptides with nine amino acids wherein two of the following criteria in is satisfied Lys at position 5; Phe at position 7; or Tyr at position 8
LMTPKQMYE

SEQ ID NO: 93 - protein, 9 aa, Artificial
  Peptides with nine amino acids wherein two of the following criteria in is satisfied Lys at position 5; Phe at position 7; or Tyr at position 8
KNKPKMNYE

SEQ ID NO: 94 - protein, 9 aa, Artificial
  Peptides with nine amino acids wherein two of the following criteria in is satisfied Lys at position 5; Phe at position 7; or Tyr at position 8
VRGPKYFYG

SEQ ID NO: 95 - protein, 9 aa, Artificial
  Peptides with nine amino acids wherein two of the following criteria in is satisfied Lys at position 5; Phe at position 7; or Tyr at position 8
AATPKIFNG

SEQ ID NO: 96 - protein, 9 aa, Artificial
  Peptides with nine amino acids wherein two of the following criteria in is satisfied Lys at position 5; Phe at position 7; or Tyr at position 8
WGEPKTWYG

SEQ ID NO: 97 - protein, 9 aa, Artificial
  Peptides with nine amino acids wherein two of the following criteria in is satisfied Lys at position 5; Phe at position 7; or Tyr at position 8
VGLKKSFYG

SEQ ID NO: 98 - protein, 9 aa, Artificial
  Peptides with nine amino acids wherein two of the following criteria in is satisfied Lys at position 5; Phe at position 7; or Tyr at position 8
LANPKEFYH

SEQ ID NO: 99 - protein, 9 aa, Artificial
  Peptides with nine amino acids wherein two of the following criteria in is satisfied Lys at position 5; Phe at position 7; or Tyr at position 8
ASSPKVAYH

SEQ ID NO: 100 - protein, 9 aa, Artificial
  Peptides with nine amino acids wherein two of the following criteria in is satisfied Lys at position 5; Phe at position 7; or Tyr at position 8
QYTPKAKYH

SEQ ID NO: 101 - protein, 9 aa, Artificial
  Peptides with nine amino acids wherein two of the following criteria in is satisfied Lys at position 5; Phe at position 7; or Tyr at position 8
QTGPKSTYI

SEQ ID NO: 102 - protein, 9 aa, Artificial
  Peptides with nine amino acids wherein two of the following criteria in is satisfied Lys at position 5; Phe at position 7; or Tyr at position 8
SMDPKRFYK

SEQ ID NO: 103 - protein, 9 aa, Artificial
  Peptides with nine amino acids wherein two of the following criteria in is satisfied Lys at position 5; Phe at position 7; or Tyr at position 8
GITPKGFYK

SEQ ID NO: 104 - protein, 9 aa, Artificial
  Peptides with nine amino acids wherein two of the following criteria in is satisfied Lys at position 5; Phe at position 7; or Tyr at position 8
RLVPKLFYK

SEQ ID NO: 105 - protein, 9 aa, Artificial
  Peptides with nine amino acids wherein two of the following criteria in is satisfied Lys at position 5; Phe at position 7; or Tyr at position 8
PSSPKVTYK

SEQ ID NO: 106 - protein, 9 aa, Artificial
  Peptides with nine amino acids wherein two of the following criteria in is satisfied Lys at position 5; Phe at position 7; or Tyr at position 8
EDEYKAFYK

SEQ ID NO: 107 - protein, 9 aa, Artificial
  Peptides with nine amino acids wherein two of the following criteria in is satisfied Lys at position 5; Phe at position 7; or Tyr at position 8
TLGPKITYQ

SEQ ID NO: 108 - protein, 9 aa, Artificial
  Peptides with nine amino acids wherein two of the following criteria in is satisfied Lys at position 5; Phe at position 7; or Tyr at position 8
EKEGKPFYQ

SEQ ID NO: 109 - protein, 9 aa, Artificial
  Peptides with nine amino acids wherein two of the following criteria in is satisfied Lys at position 5; Phe at position 7; or Tyr at position 8
VAQPKEVYR

SEQ ID NO: 110 - protein, 9 aa, Artificial
  Peptides with nine amino acids wherein two of the following criteria in is satisfied Lys at position 5; Phe at position 7; or Tyr at position 8
EAGPPAFYR

SEQ ID NO: 111 - protein, 9 aa, Artificial
  Peptides with nine amino acids wherein two of the following criteria in is satisfied Lys at position 5; Phe at position 7; or Tyr at position 8
QINPKCFYT

SEQ ID NO: 112 - protein, 9 aa, Artificial
  Peptides with nine amino acids wherein two of the following criteria in is satisfied Lys at position 5; Phe at position 7; or Tyr at position 8
DIPPKFFYT

SEQ ID NO: 113 - protein, 9 aa, Artificial
  Peptides with nine amino acids wherein two of the following criteria in is satisfied Lys at position 5; Phe at position 7; or Tyr at position 8
DDNPKTFYW

SEQ ID NO: 114 - protein, 9 aa, Artificial
  Peptides with nine amino acids wherein two of the following criteria in is satisfied Lys at position 5; Phe at position 7; or Tyr at position 8
DAVPKIEYY

SEQ ID NO: 115 - protein, 9 aa, Artificial
  Peptides with nine amino acids wherein two of the following criteria in is satisfied Lys at position 5; Phe at position 7; or Tyr at position 8
VIHEKGFYY

SEQ ID NO: 116 - protein, 9 aa, Artificial
  Peptides with nine amino acids wherein two of the following criteria in is satisfied Lys at position 5; Phe at position 7; or Tyr at position 8
GILGFVFTL

(19) United States
(12) Patent Application Publication
(10) Pub. No.: US 2007/0105779 A1
(43) Pub. Date: May 10, 2007

(54) Isolated, ssx-2 and ssx-2 related peptides useful as hla binders and ctl epitopes, and uses thereof

(76) Inventors: Danila Valmori, Lausanne (CH); Maha Ayyoub, New York, NY (US); Clemencia Pinilla, Cardiff by the Sea, CA (US)

Correspondence Address: FULBRIGHT & JAWORSKI, LLP, 666 FIFTH AVE, NEW YORK, NY, 10103-3198, US

(21) Appl. No.: 10/523,306

(22) PCT Filed: Jul. 23, 2003
(86) PCT No.: PCT/US03/23306

§ 371 Date: Sep. 28, 2005

Related U.S. Application Data
(60) Provisional application No. 60/400,076, filed on Jul. 31, 2002.

(51) Int. Cl. A61K 38/08

(52) U.S. Cl. 514/15; 514/16

(57) ABSTRACT

The invention related to peptides which bind to HLA molecules and are reactive with T cells that also react with complexes of HLA-A2 molecules and the peptide of SEQ ID NO: 17. Various uses of the peptides are disclosed.

### RELATED APPLICATION

This application is a continuation in part of Ser. No. 09/344,040 filed Jun. 25, 1999, incorporated herein by reference in its entirety.

### FIELD OF THE INVENTION

This invention relates to HLA binding peptides based upon antigens associated with cancer; especially antigens based upon the molecule referred to as SSX-2. These peptides bind to Class I molecules, and provoke lysis of the cells to which they bind, by cytolytic T lymphocytes.

### BACKGROUND AND PRIOR ART

It is fairly well established that many pathological conditions, such as infections, cancer, autoimmune disorders, etc., are characterized by the inappropriate expression of certain molecules. These molecules thus serve as “markers” for a particular pathological or abnormal condition. Apart from their use as diagnostic “targets”, i.e., materials to be identified to diagnose these abnormal conditions, the molecules serve as reagents which can be used to generate diagnostic and/or therapeutic agents. A by no means limiting example of this is the use of cancer markers to produce antibodies specific to a particular marker. Yet another non-limiting example is the use of a peptide which complexes with an MHC molecule, to generate cytolytic T cells against abnormal cells.

Preparation of such materials, of course, presupposes a source of the reagents used to generate these. Purification from cells is one laborious, far from sure method of doing so. Another preferred method is the isolation of nucleic acid molecules which encode a particular marker, followed by the use of the isolated encoding molecule to express the desired molecule.

To date, two strategies have been employed for the detection of such antigens, in e.g., human tumors. These will be referred to as the genetic approach and the biochemical approach. The genetic approach is exemplified by, e.g., dePlaen et al., Proc. Natl. Sci. USA 85: 2275 (1988), incorporated by reference. In this approach, several hundred pools of plasmids of a cDNA library obtained from a tumor are transfected into recipient cells, such as COS cells, or into antigen-negative variants of tumor cell lines which are tested for the expression of the specific antigen. The biochemical approach, exemplified by, e.g., O. Mandelboim, et al., Nature 369: 69 (1994) incorporated by reference, is based on acidic elution of peptides which have bound to MHC-class I molecules of tumor cells, followed by reversed-phase high performance liquid chromography (HPLC). Antigenic peptides are identified after they bind to empty MHC-class I molecules of mutant cell lines, defective in antigen processing, and induce specific reactions with cytotoxic T-lymphocytes. These reactions include induction of CTL proliferation, TNF release, and lysis of target cells, measurable in an MTT assay, or a ⁵¹Cr release assay.

These two approaches to the molecular definition of antigens have the following disadvantages: first, they are enormously cumbersome, time-consuming and expensive; and second, they depend on the establishment of cytotoxic T cell lines (CTLs) with predefined specificity.

The problems inherent to the two known approaches for the identification and molecular definition of antigens is best demonstrated by the fact that both methods have, so far, succeeded in defining only very few new antigens in human tumors. See, e.g., van der Bruggen et al., Science 254: 1643-1647 (1991); Brichard et al., J. Exp. Med. 178: 489-495 (1993); Coulie, et al., J. Exp. Med. 180: 35-42 (1994); Kawakami, et al., Proc. Natl. Acad. Sci. USA 91: 3515-3519 (1994).

Further, the methodologies described rely on the availability of established, permanent cell lines of the cancer type under consideration. It is very difficult to establish cell lines from certain cancer types, as is shown by, e.g., Oettgen, et al., Immunol. Allerg. Clin. North. Am. 10: 607-637 (1990). It is also known that some epithelial cell type cancers are poorly susceptible to CTLs in vitro, precluding routine analysis. These problems have stimulated the art to develop additional methodologies for identifying cancer associated antigens.

One key methodology is described by Sahin, et al., Proc. Natl. Acad. Sci. USA 92: 11810-11913 (1995). Also, see U.S. Pat. No. 5,698,396. These references are incorporated by reference. To summarize, the method involves the expression of cDNA libraries in a prokaryotic host. (The libraries are secured from a tumor sample). The expressed libraries are then immunoscreened with absorbed and diluted sera, in order to detect those antigens which elicit high titer humoral responses. This methodology is known as the SEREX method (“Serological identification of antigens by Recombinant Expression Cloning”). The methodology has been employed to confirm expression of previously identified tumor associated antigens, as well as to detect new ones. See the above referenced patent applications and Sabin, et al., supra, as well as Crew, et al., EMBO J 144: 2333-2340 (1995).

The SEREX methodology has been applied to esophageal cancer samples, an antigen has been identified, and its encoding nucleic acid molecule isolated and cloned. See, e.g., U.S. Pat. No. 5,804,381, referred to supra. The antigen and truncated forms have been found to be reactive with antibodies in the serum of cancer patients. It has also been found that peptides derived form this molecule bind with MHC molecules, provoking both cytolytic T cell and helper T cell responses. It has been found that variations of these peptides can be used as well.

The relationship between some of the tumor associated genes and a family of genes, known as the SSX genes, has been under investigation for some time. See Sahin, et al., Proc. Natl. Acad. Sci. USA 92:11810-11813 (1995); Tureci, et al., Cancer Res 56:4766-4772 (1996). One of these SSX genes, referred to as SSX-2, was identified, at first, as one of two genes involved in a chromosomal translocation event (t(X;18)(p11.2; q 11.2)), which is present in 70% of synovial sarcomas. See Clark, et al., Nature Genetics 7:502-508 (1994); Crew et al., EMBO J 14:2333-2340 (1995). It was later found to be expressed in a number of tumor cells, and is now considered to be a tumor associated antigen referred to as HOM-MEL-40 by Tureci, et al, supra. Its expression to date has been observed in cancer cells, and normal testis only. This parallels other members of the “CT” family of tumor antigens, since these are expressed only in cancer and testis cells. Crew et al. also isolated and cloned the SSX-1 gene, which has 89% nucleotide sequence homology with SSX-2. See Crew et al., supra, for information on the complete nucleotide sequences of these molecules. Additional work directed to the identification of SSX genes has resulted in the identification of SSX-3, as is described by DeLeeuw, et al., Cytogenet. Genet 73:179-183 (1996). The fact that SSX presentation parallels other, CT antigens suggested that other SSX genes might be isolated. See Gure, et al. Int. J. Cancer 72:965-971 (1997), incorporated by reference.

With respect to additional literature on the SSX family, most of it relates to SSX-1. See PCT Application W/96 02641A2 to Cooper, et al, detailing work on the determination of synovial sarcoma via determination of SSX-1 or SSX-2. Also note DeLeeuw, et al. Hum. Mol. Genet 4(6):1097-1099 (1995). also describing synovial sarcoma and SYT-SSX-1 or SSX-2 translocation. Also see Kawai, et al, N. Engl. J. Med 338(3):153-160 (1998); Noguchi, et al. Int. J. Cancer 72(6):995-1002 (1997), Hibshoosh, et al., Semin. Oncol 24(5):515-525 (1997), Shipley, et al., Am. J. Pathol. 148(2):559-567 (1996); Fligman, et al. Am. J. Pathol. 147(6); 1592-1599 (1995). Also see Chand, et al., Genomics 30(3):545-552 (1995), Brett, et al., Hum. Mol Genet 6(9):1559-1564 (1997), deBruyn, et al., Oncogene (13/3):643-648. The SSX-3 gene is described by deLeeuw, et al., Cytogenet Cell Genet 73(3):179-1983 (1966).

It is generally acknowledged that tumor antigen specific CTLs are the main effectors in the adaptive immune response against tumors. The eliciting and/or enhancement of tumor specific CTL responses in cancer patients is a primary goal of clinical trials in cancer immunotherapy. Tumor reactive CTLs recognize complexes formed by short, tumor derived peptides which are generated by intracellular processing machinery, and presented on cancer cell surfaces in association with MHC Class I molecules.

The development of vaccine strategies aimed at eliciting tumor specific CTLs, and the molecular monitoring of trials which test vaccine efficacy rely on some precise knowledge of peptide sequences.

The requirement for knowledge of peptide sequence information has led to several strategies for sequence identification.

For example, following the cloning of the gene encoding the relevant protein, one may narrow the region encoding the relevant peptide by transfecting fragments of the gene into target cells, and then testing with specific CTLs.

“Reverse immunology,” a second technique, analyzes the antigenicity and immunogenicity of synthetic peptides derived from the protein. These peptides are selected based upon their potential ability to bind to specific HLA alleles, based upon motif analysis. Yet a third approach utilized high performance liquid chromatography fractionation in combination with mass spectrometry to sequence peptides elated from tumor cell lines. These are then tested for recognition by tumor reactive CTLs.

All of these procedures are useful, but they are all also labor intensive, involve a great deal of time, and are not free from problems. As a result, the development and identification of tumor specific CTLs and epitopes has proceeded at a much lower rate than the rate at which corresponding encoding genes and proteins have developed. Design and validation of strategies which permit rapid identification of CTL epitopes is very important, in many areas of cancer research.

The technique of positional scanning of synthetic combinational peptide libraries, or “PS-SCLs” has been used to search for relevant peptides. As will be seen in the disclosure which follows, the technique has resulted in the identification of a number of relevant peptides.

### BRIEF DESCRIPTION OF THE FIGURES

FIG. 1 shows the results of a ⁵¹Cr release assay, using CTL LAU 50/4D7 on cell line Me275.

FIG. 2 compiles data relating to recognition of members of combinatorial peptide libraries.

FIGS. 3A-3D show a compilation of the data from FIG. 2 (panel A), antigenic activity of peptides identified via the PS-SCL system (panel B), data from functional competitive assays (panel C) and a compilation of data from all peptides (panel D).

FIGS. 4A-4D show the recognition of natural SSX sequences by LAU 50/4D7. Panel A shows lysis in a ⁵¹Cr release assay. Panel B shows the relative antigenicity of various homologs to SSX-2₄₁₋₄₉, which is the best binder. The code for this panel is provided in Panel D. Panel C presents the score distribution of peptides in various protein database.

### DETAILED DESCRIPTION OF PREFERRED EMBODIMENTS

### EXAMPLE 1

It has been established, by e.g., Valmori, et al., Cancer Res 50:4499-5006 (2000); Rimoldi, et al., J. Immunol 165:7253-61 (2000); and Valmori, et al., Cancer Res. 51:509-512 (2001), all of which are incorporated by reference, that metastatic, malignant melanoma lesions are an excellent source of tumor specific CTLs. The references cited supra used limiting dilution cloning techniques on a tumor infiltrated lymph node culture of a melanoma patient, to derive CTLs specific to various epitopes presented by complexes of HLA molecules and peptides.

A parallel set of experiments were carried out on a tumor infiltrated lymph node of a patient referred to as “LAU 50.” The sample had been cultured for 14 days in CTL medium containing 100 U/ml of recombinant human IL-2, and 10 ng/ml of recombinant human IL-7. Cells were then cloned via limited dilution culture in the presence of irradiated, allogeneic PBMCs, phytohemagluttinin (PHA), and recombinant human IL-2, as described by Russo. et al., Cell Immunol 88:228-232 (1984), incorporated by reference. The clones were expanded via 3-4 week restimulation in microliter plates, together with irradiated feeder cells, in the presence of PHA and recombinant human IL-2. The results of this limited dilution work was CD8⁺ T cell clone 50/4D7, which was used in experiments described infra.

### EXAMPLE 2

CTL clone LAU 50/4D7 was used in a ⁵¹Cr release assay using standard methods. In brief, cells of target cell line Me275, which is a melanoma line derived from the same patient as was LAU 50/4D7, were labeled with ⁵¹Cr for one hour at 37° C., as were control cells “T2.” This line, more accurately referred to as “CEMx721.T2,” is described by Salter, et al., Immunogenetics 21:235-248 (1985).

Cells were washed, three times, and then were incubated with the LAU 50/4D7 cells, at various effector:target ratios (0.1/1, 1/1, 10/1, 100/1) Four hours after incubation at 37° C., chromium in the supernatant was measured. Percent specific lysis was calculated as:

$$100 \times \frac{[ experimental \text{-} spontaneous release ) ]}{[ ( total \text{-} spontaneous release ) ]}$$

The CD8⁺ clone showed a high degree of lysis of the autologous, Me275 cell line, but did not lyse T2.

In a follow up experiment, Me275 cells were incubated at a 10/1 ratio, in the presence of anti HLA-A2 mAb CR 11.351. Specific lysis of the Me275 cells was inhibited in the presence of the anti HLA-A2 mAb, indicating that the T cell recognition was HLA-A2 restricted. Specifically, the T cell receptor of the T cell clone LAU 50/4D7 recognized peptide-MHC complexes on the cell surfaces of the melanoma cells consisting of an HLA-A2 molecule and a peptide derived from a protein antigen expressed in tumor cells, such as melanoma, but not normal cells.

### EXAMPLE 3

Several peptides, processed from antigenic proteins in tumor cells, such as melanoma and presented in complexes with HLA-A2 molecules on cell surfaces, are known to be recognized by tumor specific T cells. These peptides were tested to determine if LAU 50/4D7 recognized any of these.

The peptide specificity of LAU 50/4D7 was investigated, in ⁵¹Cr release assays, using twelve known HLA-A2 binders, i.e., the peptides Melan-A₂₆₋₃₅, tyrosinase₁₋₉, tyrosinase₃₆₅₋₃₇₁, gp100₁₅₁₋₁₅₇, gp100₂₀₈₋₂₁₇, gp100₂₈₀₋₂₈₈, gp100₄₅₇₋₄₆₈, gp100₄₇₅₋₄₈₅, NY-ESO-1₁₅₇₋₁₆₅, CAMEL₁₋₁₁, MAGE-A10₂₅₆₋₂₆₄, and MAGE-A4₂₂₉₋₂₃₆. In these assays, following ⁵¹Cr labeling of the HLA-A2⁺ target cells, peptides were added and then lysis was determined, as described supra. The clone failed to recognize any of these HLA-A2 presented peptides with any significance.

### EXAMPLE 4

A new approach to the identification of T cell epitopes is the combined use of tumor reactive T cell clones, and “positional scanning synthetic combinatorial libraries,” or “PS-SCLs.” See Pinilla et al. Biotechniques 13:901-5 (1992) and PCT application WO 02/22860, both incorporate by reference.

The ability of LAU 50/4D7 to recognize members of a nonapeptide PS-SCL was tested. Nonapeptides were chosen because the vast majority of optimally recognized peptides are 9 amino acids long. See, e.g., Ramensee, et al., Immunogenetics 41:178-228 (1995), incorporated by reference. The PS-SCL technique is based upon the assumption that the contribution of amino acids at each position on an epitope is independent and additive.

A library was constructed. using the format “OX8,” where “O” is one of the 20 natural L amino acids, in a defined position, while “X” may be any amino acid except Cys. This results in 180 mixtures of peptides. Each mixture contains 1.8×10¹⁰ peptides, and the entire library, including all mixtures, contains 3.1×10¹¹ nonapeptides.

The peptide libraries were screened for recognition by T cell clone LAU 50/4D7 in standard, ⁵¹Cr release assays, using T2 cells, because these express HLA-A2, and it has been established that LAU 50/4D7 recognizes HLA-A2 complexes. It is to be understood, however, that if the HLA restricting element is not known, one may substitute autologous antigen presenting cells.

Again, a ⁵¹Cr release assay was used, combining 1000 labeled T2 cells in 50 μl volume, which were incubated in the presence of 50 μl volumes of the libraries, for 15 minutes at room temperature. Effector cells were then added, (50 μl), at an effector:target ratio of 10:1. Results are presented in FIG. 1. A compilation is presented in FIG. 2. The explanation for the symbols employed is provided in that figure, when “SD” is an abbreviation for “standard deviation.”

### EXAMPLE 5

Based upon the results presented supra a series of peptides predicted to bind to HLA-A2 and to recognized by T cell clone LAU 50/4D7 were synthesized, i.e.:

| | |
|---|---|
| AAAPKIFYA | SEQ ID NO: 1 |
| AAGPKIFYA | SEQ ID NO: 2 |
| AAAAKIFYA | SEQ ID NO: 3 |
| AAAPAIFYA | SEQ ID NO: 4 |
| AAAPKAFYA | SEQ ID NO: 5 |
| AAAPKIFAA | SEQ ID NO: 6 |

T cell recognition by clone LAU 50/4D7 and binding to HLA-A2 by these peptides were determined experimentally. Peptide binding to HLA-A2 was determined in a functional competition assay. To elaborate, the functional competition assay was based upon the ability of the peptide to inhibit the binding of the tyrosinase peptide 368-376 by HLA-A*0201 restricted, specific cells, in accordance with Valmori, et al., J. Immunol 161:6958-62 (1998), incorporated by reference. T2 cells were ⁵¹Cr labeled, in the presence of anti-class I mAb W6/32, and then various concentrations of competitor peptides (50 μl volume), were added together with tyrosinase specific CTL clone LAU 156/34, at 10,000 cells/well, in 50 μl. ⁵¹Cr was measured after 4 hours of incubation at 37° C. Concentrations of competitor peptides necessary to achieve 50% inhibition of target cell lysis were calculated, and used to determine binding values.

To determine T cell recognition of the peptides, standard ⁵¹Cr release assays were performed using T2 cells pulsed with serial dilutions of each peptide. The peptide dose required to induce half maximal lysis was determined.

FIGS. 3 and 4 present these results. The relative recognition and binding of the peptides is as follows:

| Peptide | Relative Recognition | Relative Binding |
|---|---|---|
| SEQ ID NO: 1 | 1 | 1 |
| SEQ ID NO: 2 | 0.01 | 0.07 |
| SEQ ID NO: 3 | 0.1 | 0.16 |
| SEQ ID NO: 4 | <10⁻⁵ | 0.9 |
| SEQ ID NO: 5 | 0.05 | 0.1 |
| SEQ ID NO: 6 | 10⁻⁵ | 0.2 |
| SEQ ID NO: 7 | <10⁻⁵ | 0.2 |

Additional peptides as well as some of the peptides discussed supra were tested, in similar assays. The results are summarized as follows:

| | | |
|---|---|---|
| | Relative | Relative |
| Peptide | Recognition | Binding |

| | | | |
|---|---|---|---|
| AAAAKIFYA | (SEQ ID NO: 3) | 0.1 | 0.16 |
| AAAPAIFYA | (SEQ ID NO: 4) | <10⁻⁶ | 0.9 |
| AAAPKAFYA | (SEQ ID NO: 5) | 0.06 | 0.1 |
| AAAPKIFAA | (SEQ ID NO: 6) | <10⁻⁵ | 0.2 |
| AADPKIFYA | (SEQ ID NO: 8) | 0.04 | 0.1 |
| AADGKIFYA | (SEQ ID NO: 9) | 0.07 | 0.07 |
| AADEKIFYA | (SEQ ID NO: 10) | 0.01 | 0.1 |
| AAGGKIFYA | (SEQ ID NO: 11) | 0.01 | 0.02 |
| AAGEKIFYA | (SEQ ID NO: 12) | 0.07 | 0.1 |
| ALAPKIFYA | (SEQ ID NO: 13) | 0.26 | 5 |
| AMAPKIPYA | (SEQ ID NO: 14) | 0.4 | 1 |
| AAAPKIFYL | (SEQ ID NO: 15) | 0.7 | 0.13 |
| AAAPKIAYA | (SEQ ID NO: 16) | 10⁻⁵ | 0.1 |

What is striking about these results is that all of the deduced peptides were specifically, and efficiently, recognized by the CTL clone. The most active of the sequences was, in fact SEQ ID NO: 1.

When amino acids known as major anchors for binding to HLA-A*0201 were added, the efficiency of peptide recognition was not improved.

Additional peptides based upon the amino acid sequence of SEQ ID NO: 1 were prepared, by substituting Ala for each of the amino acids at positions 4, 5, 6, 7 and 8 one at a time, and were analyzed in similar fashion. The substitutions at positions 5, 7 and 8 impaired recognition dramatically, while substitution at 4 and 6 resulted in more limited reduction. Notwithstanding this, the reduction of binding to HLA-A2 never dropped more than 10 fold.

### EXAMPLE 6

The optimal sequence set forth supra, i.e., SEQ ID NO: 1, bore many similarities to a peptide identified previously as an HLA-A2 binder, derived from the protein known as SSX-2. The peptides is KASEKIFYV (SEQ ID NO: 17), as described in, e.g., PCT Application PCT/US99/14495, published Jan. 6, 2000, incorporated by reference. SEQ ID NO: 1 and SEQ ID NO: 17 share residues at positions 2 and 5-8. Further, the most active mixtures tested in the PS-SCL analysis reported supra also shared these positions. The single most active mixture, however, had residues P, G and E at positions 4, 5 and 6. Position 6 (E) is the native amino acid. The observations are comparable with prior showings that not all amino acids which prove to be most active are those which are present in the natural sequence. See Pinilla, et al., Cancer Res 61:5153-5160 (2001), incorporated by reference.

In view of these prior observations and the similarities, the peptide of SEQ ID NO: 17 was tested in a ⁵¹Cr release assay, as described herein, using LAU 50/4D7. The sequence was recognized, and what is remarkable about the recognition is that the efficiency of recognition is in the same range as that of SEQ ID NO: 1.

In order to further substantiate the recognition and efficiency of lysis, LAU 50/4D7 was tested for its lytic ability on SK-MEL-37, a melanoma cell line which expresses both SSX-2 and HLA-A*0201. The CD8+ clone lysed SK-MEL-37 efficiently; however, it did not lyse either SK-MEL-23, which expresses HLA-A2 but not SSX-2, or Me260 which expresses SSX-2, but not HLA-A2. Further, the recognition of endogenously produced complexes of HLA-A2 and the peptide of SEQ ID NO: 17 was confirmed, directly, by measuring T cell production of gamma interferon, in response to stimulation with COS7 cells that had been transiently cotransfected with plasmids encoding SSX-2 and HLA-A*0201.

### EXAMPLE 7

In addition to the peptides described supra, a set of peptides were synthesized, based upon the sequence of SEQ ID NO: 17. These peptides were tested for their ability to bind HLA-A2, as well as their recognition by LAU 50/4D7. Experiments were carried out as described, supra, using peptides SEQ ID NOS: 18-23 which are based upon this peptide. The results, together with results obtained with SEQ ID NOS: 1-17, are presented herein:

| | | | | | |
|---|---|---|---|---|---|
| | | 50% | | | |
| | SEQ | maximal | Relative | 50% | |
| Peptide | ID | lysis | recogni- | inhibition | Relative |
| Sequence | NO: | [pM] | tion | [nM] | binding |

| | | | | | |
|---|---|---|---|---|---|
| KASEKIFYV | 17 | 60 | 1 | 200 | 1 |
| AAAPKIFYA | 1 | 20 | 3 | 20 | 10 |
| AADPKIFYA | 8 | 600 | 0.1 | 20 | 1 |
| AADGKIFYA | 9 | 300 | 0.2 | 286 | 0.7 |
| AADEKIFYA | 10 | 2000 | 0.03 | 20 | 1 |
| AAGPKIFYA | 2 | 2000 | 0.03 | 286 | 0.7 |
| AAGGKIFYA | 11 | 2000 | 0.03 | 1000 | 0.2 |
| AAGEKIFYA | 12 | 300 | 0.2 | 200 | 1 |
| ALAPKIFYA | 13 | 75 | 0.8 | 4 | 50 |
| AMAPKIFYA | 14 | 50 | 1.2 | 20 | 10 |
| AAAPKIFYL | 15 | 29 | 2.1 | 154 | 1.3 |
| AAAAKIFYA | 3 | 200 | 0.3 | 133 | 1.5 |
| AAAPAIFYA | 4 | >10⁵ | <10⁻⁵ | 22 | 9 |
| AAAPKAFYA | 5 | 300 | 0.2 | 200 | 1 |
| AAAPKIAYA | 16 | 10⁵ | 10⁻⁵ | 200 | 1 |
| AAAPKIFAA | 6 | >10⁵ | <10⁻⁵ | 100 | 2 |
| AASEKIFYV | 18 | 60 | 1 | nd | Na |
| KAAEKIFYV | 19 | 60 | 1 | nd | Na |
| KASAKIFYV | 20 | 300 | 0.2 | nd | Na |
| KASEAIFYV | 21 | >10⁵ | <10⁻⁵ | nd | Na |
| KASEKAFYV | 22 | 300 | 0.2 | nd | Na |
| KASEKIAYV | 23 | 1000 | 0.06 | nd | Na |
| KASEKIFAV | 24 | >10⁵ | <10⁻⁵ | nd | Na |
| KASEKIFYA | 25 | 11 | 5.4 | nd | Na |

The peptides which are presented in bold (SEQ ID NOS: 1, 15, 13, 14, 18, 19 and 23) all show levels of T cell recognition at least equal to the natural peptide, i.e., SEQ ID NO: 17.

### EXAMPLE 8

An additional group of peptides were synthesized, which consisted of nine amino acids, and satisfied two the following criteria
Lys at position 5
Phe at position 7
Tyr at position 8

These peptides were tested in lysis assays, using the T cell clone LAU50/4D7, referred to supra. In these assays, T2 cells were used as the target, and were incubated with each of the listed peptides, at a concentration of 1 μM, at 37° C. for 1 hour. The T2 cells were then washed to remove excess peptides, and the T cells were incubated with the peptide pulsed target T2 cells for 4 hours at 37° C. Percentage lysis was determined, and the results follows:

| | | |
|---|---|---|
| PEPTIDE SEQUENCE | % | SEQ ID |

| | | | | | | | | | | |
|---|---|---|---|---|---|---|---|---|---|---|
| 1 | 2 | 3 | 4 | 5 | 6 | 7 | 8 | 9 | Lysis | NO. |

| | | | | | | | | | | |
|---|---|---|---|---|---|---|---|---|---|---|
| -*A | A | A | P | K | Q | F | L | A | 70 | 60 |
| A | A | S | P | K | S | F | T | L | 72 | 61 |
| V | S | A | P | K | V | F | Q | A | 75 | 62 |
| I | A | S | P | K | A | T | Y | V | 68 | 63 |
| V | S | A | P | K | I | F | Q | A | 66 | 64 |
| N | S | L | P | K | V | A | Y | A | 68 | 65 |
| A | S | L | P | K | V | S | Y | V | 65 | 66 |
| K | A | E | P | K | A | P | Y | A | 66 | 67 |
| H | S | L | P | K | V | S | Y | A | 80 | 68 |
| T | A | S | P | K | E | F | Y | A | 73 | 69 |
| A | S | V | P | K | E | L | Y | L | 75 | 70 |
| S | P | D | P | K | I | C | Y | V | 73 | 71 |
| V | A | E | P | K | E | S | Y | V | 73 | 72 |
| I | S | A | P | K | I | F | R | V | 60 | 73 |
| A | V | Y | P | K | I | F | Y | V | 50 | 74 |
| K | A | S | E | K | I | I | Y | V | 73 | 75 |
| V | Q | D | P | K | V | T | Y | L | 75 | 76 |
| C | P | V | P | K | I | F | Y | V | 55 | 77 |
| F | A | K | P | K | I | T | Y | V | 80 | 78 |
| Q | T | P | P | K | I | D | Y | L | 65 | 79 |
| K | A | S | E | K | I | F | Y | V | 68 | 17 |
| L | R | S | P | K | L | F | Y | A | 60 | 80 |
| K | Y | S | E | K | I | S | Y | V | 40 | 81 |
| E | Q | K | P | K | D | F | Y | A | 60 | 82 |
| R | T | T | P | K | D | F | Y | V | 45 | 83 |
| R | A | D | P | K | H | K | Y | L | 50 | 80 |
| K | R | G | W | K | T | F | Y | A | 48 | 85 |
| T | S | S | W | K | K | F | Y | L | 65 | 86 |
| L | S | R | P | K | R | F | Y | L | 60 | 87 |
| N | T | Y | P | K | G | F | Y | C | 5 | 88 |
| S | K | L | P | K | D | F | Y | D | 1 | 89 |
| A | L | S | P | K | E | F | Y | E | 4 | 90 |
| K | V | D | P | K | P | F | Y | E | 8 | 91 |
| V | G | S | E | K | L | F | Y | E | 6 | 92 |
| L | M | T | P | K | Q | M | Y | E | 4 | 93 |
| K | N | K | P | K | M | N | Y | E | 5 | 94 |
| V | R | G | P | K | Y | F | Y | G | 15 | 95 |
| A | A | T | P | K | I | F | N | G | 50 | 96 |
| W | G | E | P | K | T | W | Y | G | 3 | 97 |
| V | G | L | K | K | S | F | Y | G | 3 | 98 |
| L | A | N | P | K | E | F | Y | H | 25 | 99 |
| A | S | S | P | K | V | A | Y | H | 6 | 100 |
| Q | Y | T | P | K | A | K | Y | H | 65 | 101 |
| Q | T | G | P | K | S | T | Y | I | 73 | 102 |
| S | M | D | P | K | R | F | Y | K | 55 | 103 |
| G | I | T | P | K | G | F | Y | K | 1 | 104 |
| R | L | V | P | K | L | F | Y | K | 7 | 105 |
| P | S | S | P | K | V | T | Y | K | 1 | 106 |
| E | D | E | Y | K | A | F | Y | K | 2 | 107 |
| T | L | G | P | K | I | T | Y | Q | 50 | 108 |
| E | K | E | G | K | P | F | Y | Q | 15 | 109 |
| V | A | Q | P | K | E | V | Y | R | 3 | 110 |
| E | A | G | P | P | A | F | Y | R | 15 | 111 |
| Q | I | N | P | K | C | F | Y | T | 20 | 112 |
| D | I | P | P | K | F | F | Y | T | 5 | 113 |
| D | D | N | P | K | T | F | Y | W | 5 | 114 |
| D | A | V | P | K | I | E | Y | Y | 1 | 115 |
| V | I | H | B | K | G | F | Y | Y | 3 | 116 |
| K | A | S | E | K | I | F | Y | V | 70 | 17 |
| G | I | L | G | F | V | F | T | L | 4 | 117 |
| G | L | Y | D | G | M | E | H | L | 2 | 35 |

SEQ ID NOS: 60-87, 96, 101-103 and 108 were all recognized by the clone with a lysis percentage greater than 40, indicating cross reactivity. The peptides of SEQ ID NOS: 17, 35 and 117 were used as negative controls. SEQ ID NO: 35 derives from MAGE-A10, while SEQ ID NO: 117 is a well known influenza matrix, or “Fluma” peptide.

The foregoing examples therefore describe various features of the invention, including, inter alia, isolated peptides which, when complexed to an HLA-A2 molecule, present a complex which is recognized by a cytolytic T cell which recognizes complexes of said HLA-A2 molecule and SEQ ID NO: 17. Preferred, but by no means limiting examples of such peptides, are the peptides of SEQ ID NO: 1, 13, 14, 15, 18, and 25, although it will be clear to the skilled artisan that other peptides can be identified using the methods described herein. As noted, e.g., one can develop CTLs specific to the HLA-A2/SEQ ID NO: 17 complexes and, with these in hand, can then screen peptides via any of the methods known to the skilled artisan including the peptide library screening methodologies utilized in the examples. Preferred are nonapeptides which have the motif described in example 8, i.e., consist of nine or more amino acids, where two of the following are satisfied: Lys at position 5, Phe at position 7, and Tyr at position 8. Also, preferred are peptides which have the “core” structure of EKIFY at positions 4-8, with the proviso that the peptide is not the peptide of SEQ ID NO: 17.

Also a part of the invention are the remaining peptides described herein, i.e., any and all of SEQ ID NOS: 2-12, 16, 17, 19-24, 26 and 117. While these peptides do not possess all of the properties of the preferred peptides, it should be noted that all possess the ability to bind HLA-A2 molecules, and perform to different degrees of success, as shown supra.

The peptides are useful, inter alia, for identifying cells which present HLA-A2 molecules on their surface, as well as for identifying relevant CTLs. One can use various methods including FACS, perhaps combined with the use of tetrameric peptide constructs, to identify and to purify desired CTLs. Such tetrameric complexes are known to the art, and comprise, e.g., an avidin or streptavidin molecule bound to four biotin molecules, which are in turn bound to complexes of HLA-A2 molecules and the peptide. Such tetrameric complexes can, e.g., be immobilized on solid surfaces and be used to remove relevant CTLs from mixed cell populations. Similarly, they can be used to stimulate the T cell populations either before or after their purification.

The ability of the peptides to form recognizable complexes makes them useful as therapeutic agents in conditions such as cancer, where the peptide forms a complex with the HLA molecule, leading to recognition by a CTL, and lysis thereby. As was shown, supra, CTLs which recognize the complexes occur naturally in patients, so administration of one or more of the peptides of the invention to a subject in need of a cytolytic T cell response is another feature of the invention. Such subjects may be, e.g., cancer patients, such as melanoma patients. Such patients may receive one of the peptides of the invention, or “cocktails” which comprise more than one peptide. The peptide component of such cocktails may consist of the peptides described herein, or may combine some peptides disclosed herein with other peptides known in the art, such as the following, which bind to Class I or Class II MHC.

| | | | SEQ |
|---|---|---|---|
| | | | ID |
| PEPTIDE SEQUENCE | ANTIGEN | HLA | NO: |
| YMDGTMSQV | TYROSINASE | A2 | 26 |
| MLLAVLYCL | TYROSINASE | A2 | 27 |
| ELAGIGILTV | MELAN-A | A2 | 28 |
| IMPKAGLLI | MAGE-A3 | A2 | 29 |
| FLWGPRALV | MAGE-A3 | A2 | 30 |
| VRIGHLYIL | MAGE-A6 | Cw7 | 31 |
| YLQLVFGLEV | MAGE-A2 | A2 | 32 |
| FLWGPRALV | MAGE-A12 | A2 | 33 |
| VLPDVFIRC(V) | GnTV | A2 | 34 |
| KASPKIFYV | SSX2 | A2 | 17 |
| GLYDGMEHL | MAGE-A10 | A2 | 35 |
| MEVDPIGHLY | MAGE-A3 | B18, B44 | 36 |
| EVDPIGHLY | MAGE-A3 | A1 | 37 |
| SLLMWITQC | NY-ESO-1 | A2 | 38 |
| IMPKAGLLI | MAGE-A3 | A24 | 39 |
| EVDPIGHLY | MAGE-A3 | B35 | 40 |
| GVYDGREHTV | MAGE-A4 | A2 | 41 |
| EADPTGHSY | MAGE-A1 | A1, B35 | 42 |
| SEIWRDIDF | TYROSINASE | B44 | 43 |
| LPSSADVEF | TYROSINASE | B35 | 44 |
| SAYGBPRKL | MAGE-A1 | Cw3, Cw6, | 45 |
| | | Cw16 | |
| YRPRPRRY | GAGE-1,2,8 | Cw6 | 46 |
| LAMPFATPM | NY-ESO-1 | Cw3 | 47 |
| ARGPESRLL | NY-ESO-1 | Cw6 | 48 |
| YYWPRPRRY | GAGE-3,4,5,6,7 | A29 | 49 |
| AARAVFLAL | BAGE-1 | Cw16 | 50 |
| TQHFVQENYLEY | MAGE-A3 | DP4 | 51 |
| SLLMWITQCFL | NY-ESO-1 | DP4 | 52 |
| AELVHFLLLKYRAR | MAGE-A3 | DR13 | 53 |
| LLKYRAREPVTKAE | MAGE-A3,A6,A2 | DR13 | 54 |
| AELVHFLLLKYRAR | MAGE-A-12 | DR13 | 55 |
| EYVIKVSARVRF | MAGE-A1 | DR15 | 56 |
| LLKYRAREPVTKAE | MAGE-A1 | DR13 | 57 |
| PGVLLKEFTVSGNILTIRLT | NY-ESO-1 | DR4 | 58 |
| AADHRQLQLSISSCLQQL | NY-ESO-1 | DR4 | 59 |

In an especially preferred embodiment, one administers a cocktail of peptides based upon the HLA profile of the subject being treated. Based upon known Class I peptide binding motifs, such as those set forth by Ramensee, et al., supra, peptides such as those set forth at SEQ ID NOS: 1-31 would be expected to bind to other HLA-Class I alleles, such as HLA-A1, A3, B7, B8, B15, B27, B44, B51 in addition to HLA-A2, and subtypes thereof. Further, if appropriate, one or more peptides which bind to HLA-A2, HLA-B7, HLA-Cw6, and so forth, can be admixed, preferably in the presence of an adjuvant like GM-CSF, alum, or another adjuvants well known to the art, such as CPG. See U.S. Pat. Nos. 6,339,068; 6,239,116; 6,207,646 and 6,194,388, all of which are incorporated by reference. Such combinations of peptides, in the form of compositions, are another feature of the invention, either alone or in combination with such adjuvants.

Yet a further feature of the invention are nucleic acid molecules which consist of nucleotide sequences that encode the peptides of the invention. Such nucleic acid molecules may be used to encode the peptides of the invention, and may be combined into expression vectors, operably lined to a promoter. More than one sequence can be combined in such expression vectors, as can nucleic acid molecules which encode HLA-A2 molecules. The constructs can be used to transfect cells, so as to generate the CTLs, or for administration to subjects in need of a cytolytic T cell response or augmenting of a pre-existing T cell response. Such administration could be one of, e.g., administering vector constructs as described, heterologous expression vectors, peptides or recombinant proteins, such as the full length proteins, preferably in recombinant form, from which one or more of the peptides are derived as discussed supra.

Other features of the invention will be clear to the skilled artisan, and need not be reiterated herein.

We claim

1. An isolated peptide which binds to an MHC molecule to form a complex that is recognized by a cytolytic T cell which recognizes and lyses cells presenting complexes of HLA-A2 molecules and the peptide whose amino acid sequence consists of SEQ ID NO: 17, with the proviso that said peptide is not the peptide of SEQ ID NO: 17.

2. The isolated peptide of claim 1, wherein the amino acid sequence of said peptide consists of an amino acid sequence found in a naturally occurring protein.

3. The isolated peptide of claim 1, wherein the amino acid sequence of said peptide consists of a non-naturally occurring amino acid sequence.

4. The isolated peptide of claim 1, consisting of 9 amino acids and satisfactory at least two of the following criteria: Lys at position 5, Phe at position 7, and Tyr at position 8.

5. The isolated peptide of claim 1, consisting of nine amino acids, wherein the amino acids at positions 4-8 are EKIFY.

6. The isolated peptide of claim 1, wherein said peptide is selected from the group consisting of SEQ ID NO: 1, 2, 3, 4, 5, 6, 7, 8, 9, 10, 11, 12, 13, 14, 15, 16, 18, 19, 20, 22, 23, 24, 25, 60-87, 96, 101-103, and 108.

7. The isolated peptide of claim 6, selected from the group consisting of SEQ ID NO: 1, 13, 14, 15, and 25.

8. A composition comprising at least two peptides of claim 1.

9. The composition of claim 8, further comprising an adjuvant.

10. A composition comprising the isolated peptide of claim 1, and at least one additional peptide.

11. The composition of claim 10, wherein said peptide consists of the amino acid sequence of any of SEQ ID NOS: 1, 13, 14, 15 or 25.

12. The composition of claim 9, wherein said at least one additional peptide binds to an HLA molecule other than HLA-A2.

13. A tetrameric molecule comprising an avidin or streptavidin molecule, bound to four biotin molecules, each of which is bound to a complex of an HLA molecule and the peptide of claim 1.

14. An isolated nucleic acid molecule consisting of a nucleotide sequence which encodes the peptide of claim 1.

15. An isolated nucleic acid molecule consisting of a nucleotide sequence which encodes the peptide of claim 1.

16. Expression vector comprising the isolated nucleic acid molecule of claim 14, operably linked to a promoter.

17. Expression vector comprising the isolated nucleic acid molecule of claim 14, operably linked to a promoter.

18. Recombinant cell comprising the isolated nucleic acid molecule of claim 13.

19. Recombinant cell comprising the expression vector of claim 16.

20. Recombinant cell comprising the expression vector of claim 17.

21. Expression vector which encodes at least two of the peptides of claim 1.

22. A method for treating a subject suffering from a pathological condition comprising administering the isolated peptide of claim 1 to said subject, in an amount sufficient to alleviate said condition.

23. The method of claim 21, wherein said pathological condition is cancer.

24. The method of claim 22, wherein said cancer is melanoma

25. A method for determining if a subject suffers from a pathological condition, comprising assaying a sample taken from said subject to determine if said sample contains cytolytic T cells which react with a complex of an HLA molecule and the peptide of claim 1, wherein presence of said cytolytic T cells is indication of said pathological condition.
